(19) United States
(12) Patent Application Publication
(10) Pub. No.: US 2018/0153398 A1
(43) Pub. Date: Jun. 7, 2018

(54) EYEBALL OPTICAL MEASUREMENT APPARATUS AND EYEBALL OPTICAL MEASUREMENT METHOD

(71) Applicant: FUJI XEROX CO., LTD., Tokyo (JP)

(72) Inventors: Junichiro HAYAKAWA, Kanagawa (JP); Kouhei YUKAWA, Kanagawa (JP); Kazuyuki MATSUSHITA, Kanagawa (JP)

(73) Assignee: FUJI XEROX CO., LTD., Tokyo (JP)

(21) Appl. No.: 15/793,355

(22) Filed: Oct. 25, 2017

(30) Foreign Application Priority Data
Dec. 6, 2016 (JP) 2016-236416

(51) Int. Cl. A61B 3/117; A61B 3/00; A61B 5/145

(52) U.S. Cl. CPC A61B 3/117; A61B 3/0041; A61B 5/1455; A61B 3/0025; A61B 5/14532

(57) ABSTRACT

An eyeball optical measurement apparatus includes a light radiating unit that radiates light toward an anterior chamber of an eyeball of a measurement subject; a light receiving unit that receives the light that has passed through the anterior chamber; an eyeball observation unit that observes an optical path relative to the eyeball while the light is radiated from the light radiating unit; and a controller that controls the light radiating unit so as to change a position at which the eyeball is irradiated with the light on the basis of a result of the observation of the eyeball by the eyeball observation unit.

### CROSS-REFERENCE TO RELATED APPLICATIONS

This application is based on and claims priority under 35 USC 119 from Japanese Patent Application No. 2016-236416 filed Dec. 6, 2016.

### Background

### Technical Field

The present invention relates to an eyeball optical measurement apparatus and an eyeball optical measurement method.

### SUMMARY

According to an aspect of the invention, there is provided an eyeball optical measurement apparatus including a light radiating unit that radiates light toward an anterior chamber of an eyeball of a measurement subject; a light receiving unit that receives the light that has passed through the anterior chamber; an eyeball observation unit that observes an optical path relative to the eyeball while the light is radiated from the light radiating unit; and a controller that controls the light radiating unit so as to change a position at which the eyeball is irradiated with the light on the basis of a result of the observation of the eyeball by the eyeball observation unit.

### BRIEF DESCRIPTION OF THE DRAWINGS

Exemplary embodiments of the present invention will be described in detail based on the following figures, wherein:

FIGS. 1A and 1B illustrate an exemplary structure of an eyeball optical measurement apparatus according to a first exemplary embodiment, where FIG. 1A shows a top view of an eyeball (sectional view along a plane perpendicular to the up-down direction) and FIG. 1B shows a front view of the eyeball;

FIG. 2 is a schematic diagram illustrating image data output by an eyeball observation unit when an eyeball is observed from the front while an optical path passes through an anterior chamber;

FIG. 3 is a flowchart of an example of an eyeball optical measurement method;

FIGS. 4A and 4B illustrate an exemplary structure of an eyeball optical measurement apparatus according to a second exemplary embodiment, where FIG. 4A shows a top view of an eyeball (sectional view along a plane perpendicular to the up-down direction) and FIG. 4B shows a front view of the eyeball;

FIGS. 5A to 5C illustrate image data obtained by the eyeball observation unit, where FIG. 5A illustrates the case where the optical path is shifted forward relative to the anterior chamber of the eyeball, FIG. 5B illustrates the case where the optical path passes through the anterior chamber, and FIG. 5C illustrates the case where the optical path is shifted backward relative to the anterior chamber of the eyeball;

FIGS. 6A and 6B illustrate an exemplary structure of an eyeball optical measurement apparatus according to a third exemplary embodiment, where FIG. 6A shows a top view of an eyeball (sectional view along a plane perpendicular to the up-down direction) and FIG. 6B shows a front view of the eyeball;

FIGS. 7A and 7B illustrate an exemplary structure of an eyeball optical measurement apparatus according to a fourth exemplary embodiment, where FIG. 7A shows a top view of an eyeball (sectional view along a plane perpendicular to the up-down direction) and FIG. 7B shows a front view of the eyeball;

FIGS. 8A and 8B illustrate an exemplary structure of an eyeball optical measurement apparatus according to a fifth exemplary embodiment, where FIG. 8A shows a top view of an eyeball (sectional view along a plane perpendicular to the up-down direction) and FIG. 8B shows a front view of the eyeball;

FIG. 9 illustrates a method by which an optical measurement apparatus measures an angle of rotation of a plane of polarization (optical rotation) due to an optically active substance contained in aqueous humor in the anterior chamber;

FIGS. 10A and 10B illustrate an exemplary structure of an eyeball optical measurement apparatus according to a sixth exemplary embodiment, where FIG. 10A shows a top view of an eyeball (sectional view along a plane perpendicular to the up-down direction) and FIG. 10B shows a front view of the eyeball;

FIGS. 11A and 11B illustrate an exemplary structure of an eyeball optical measurement apparatus according to a seventh exemplary embodiment, where FIG. 11A shows a top view of an eyeball (sectional view along a plane perpendicular to the up-down direction) and FIG. 11B shows a front view of the eyeball; and

FIGS. 12A and 12B illustrate an exemplary structure of an eyeball optical measurement apparatus according to an eighth exemplary embodiment, where FIG. 12A shows a top view of an eyeball (sectional view along a plane perpendicular to the up-down direction) and FIG. 12B shows a front view of the eyeball.

### DETAILED DESCRIPTION

Exemplary embodiments of the present invention will hereinafter be described with reference to the accompanying drawings. In the drawings, an eyeball may be drawn larger or smaller than other components (for example, an optical system described below) to clarify the relationship between the eyeball and an optical path.

### Background of Measurement of Glucose Concentration in Aqueous Humor

The background of measurement of glucose concentration in aqueous humor will be described.

Type-1 and type-2 diabetics (measurement subjects), who require insulin therapy, are recommended to self-measure their blood glucose levels. The measurement subjects measure their own blood glucose levels at, for example, their homes to achieve precise blood glucose control.

A currently available self-blood glucose measurement device measures glucose concentration in blood by using a small amount of blood obtained by, for example, pricking a fingertip with a needle. Self-blood glucose measurement is often recommended to be performed after each meal or before going to bed, for example, and needs to be performed once to several times a day. More frequent measurements are necessary in intensive insulin therapy.

Therefore, an invasive blood glucose measurement method using a self-blood glucose measurement device that involves skin puncturing often discourages the measurement subject to self-measure their blood glucose level because of the pain caused when blood is collected (at the time of blood collection). This makes it difficult to achieve efficient diabetes treatment.

Accordingly, non-invasive blood glucose measurement methods that do not require skin puncturing are being developed as an alternative to the invasive blood glucose measurement method that involves skin puncturing.

Examples of non-invasive blood glucose measurement methods that are being studied include methods using near infrared spectroscopy and photoacoustic spectroscopy and a method utilizing optical activity. In these methods, the blood glucose level is estimated from glucose concentration.

In the methods using near infrared spectroscopy and photoacoustic spectroscopy, an optical absorption spectrum or acoustic vibration of blood in blood vessels of a finger is detected. However, since blood contains cell substances, such as erythrocytes and leukocytes, these methods are greatly influenced by light scattering. In addition, these methods are influenced not only by the blood in the blood vessels but also by the surrounding tissue. Accordingly, in these methods, a signal regarding the glucose concentration needs to be extracted from a signal affected by a large number of substances, such as protein and amino acid. The signal separation cannot be easily performed.

In contrast, aqueous humor contained in an anterior chamber has substantially the same composition as that of serum, and contains protein, glucose, and ascorbic acid. Unlike blood, aqueous humor does not contain cell substances, such as erythrocytes and leukocytes, and is less influenced by light scattering. Therefore, aqueous humor is suitable for an optical measurement of glucose concentration.

Accordingly, aqueous humor may be used to optically measure the concentration of optically active substances including glucose.

Since aqueous humor is tissue fluid that transports glucose, the glucose concentration in the aqueous humor is considered to be correlated with the glucose concentration in the blood. According to a report of measurements performed on rabbits, the time required to transport glucose from the blood to the aqueous humor (transportation delay time) is within 10 minutes.

As described above, the glucose concentration in the blood may be determined by measuring the glucose concentration in the aqueous humor.

In a method for optically measuring the concentration of optically active substances, such as glucose, contained in the aqueous humor, the following two optical paths may be set.

One optical path is a path along which light is incident on the eyeball in a direction nearly perpendicular to the eyeball surface, that is, in the front-back direction, reflected by the interface between the cornea and the aqueous humor or the interface between the aqueous humor and the lens, and then received. The other optical path is a path along which light is incident on the eyeball in a direction nearly parallel to the eyeball surface, transversely transmitted through (caused to pass through) the anterior chamber, and then received.

The first optical path, along which light is incident on the eyeball in a direction nearly perpendicular to the eyeball surface, has a risk that the light will reach the retina. In particular, when the light source is a highly coherent laser, there is a risk that the light will reach the retina.

In contrast, the second optical path, along which light is incident on the eyeball in a direction nearly parallel to the eyeball surface and transversely transmitted through the anterior chamber, does not easily cause the light to reach the retina.

The concentration and optical activity of optically active substances depend on the length of the optical path, and the optical rotation increases as the length of the optical path increases. When the light is transversely transmitted through the anterior chamber, the length of the optical path is increased.

For the above-described reasons, the optical path along which the light is transversely transmitted through the anterior chamber is employed herein.

The concentration of the optically active substances including glucose may be determined from the intensity of the light transmitted through the anterior chamber.

Protein, glucose, ascorbic acid, etc., contained in the aqueous humor are optically active substances, and exhibit optical activity. Therefore, the concentration of the optically active substances including glucose may be optically measured by utilizing the optical activity thereof.

The optical measurement of the concentration of the optically active substances contained in the aqueous humor in the anterior chamber of the eyeball is referred to as “eyeball optical measurement” or “optical measurement” herein.

### First Exemplary Embodiment

### Optical Measurement Apparatus 1

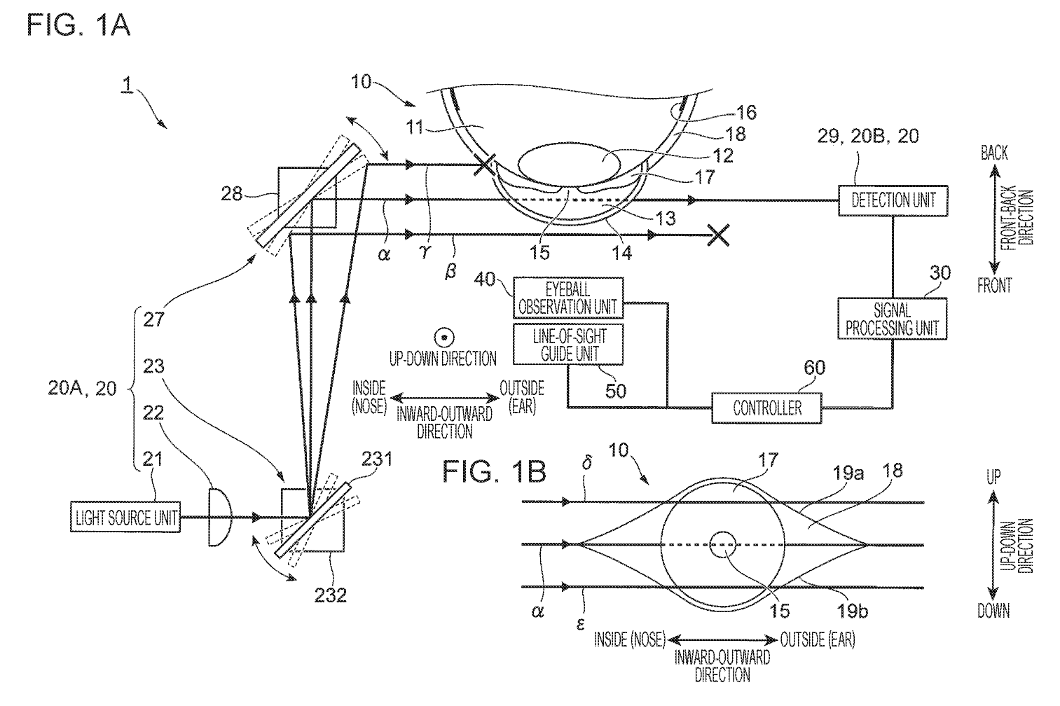

FIGS. 1A and 1B illustrate an exemplary structure of an eyeball optical measurement apparatus 1 according to a first exemplary embodiment. FIG. 1A shows a top view of an eyeball 10 (sectional view along a plane perpendicular to the up-down direction), and FIG. 1B shows a front view of the eyeball 10. Assume that the eyeball 10 illustrated in FIGS. 1A and 1B is a left eyeball. In FIGS. 1A and 1B, the inward-outward direction, which is the direction between the inner region (nose) and the outer region (ear) of the face, the front-back direction, which is the direction between the front and back of the face, and the up-down direction, which is the direction between the top and bottom of the face, are indicated by the respective signs such as arrows.

The eyeball optical measurement apparatus 1 (hereinafter sometimes referred to as an optical measurement apparatus 1) includes an optical system 20, a signal processing unit 30, an eyeball observation unit 40, a line-of-sight guide unit 50, and a controller 60.

The optical system 20 radiates light toward an anterior chamber 13 (described below) of the eyeball (test object) 10 of a measurement subject (test subject), and receives the light transmitted through the anterior chamber 13. The signal processing unit 30 processes a signal obtained by the optical system 20. The eyeball observation unit 40 observes an optical path relative to the eyeball 10, and acquires image data showing the positional relationship between the eyeball 10 and the optical path. The line-of-sight guide unit 50 guides the line of sight of the measurement subject (eyeball 10) so that the line of sight extends in a predetermined direction. The controller 60 controls the optical system 20, the signal processing unit 30, the eyeball observation unit 40, and the line-of-sight guide unit 50.

It is not necessary that the optical measurement apparatus 1 include the line-of-sight guide unit 50.

The optical measurement apparatus 1 according to the first exemplary embodiment measures the concentration of an optically active substance contained in the aqueous humor based on the intensity of light transmitted through the aqueous humor.

The structure of the eyeball 10 will now be described.

As illustrated in FIG. 1A, the eyeball 10 has a substantially spherical external shape, and includes a vitreous body 11 at the center. In FIG. 1A, the posterior half of the eyeball 10 is omitted. A crystalline lens 12, which functions as a lens, is embedded in a portion of the vitreous body 11. The anterior chamber 13 is disposed in front of the crystalline lens 12, and a cornea 14 is disposed in front of the anterior chamber 13. The anterior chamber 13 and the cornea 14 are convex and project from the spherical body.

The crystalline lens 12 is surrounded by an iris 17 at the periphery thereof, and a pupil 15 is provided at the center of the iris 17. A portion of the vitreous body 11 excluding a portion that is in contact with the crystalline lens 12 is covered with a retina 16. The retina 16 is covered with a sclera 18. Thus, the eyeball 10 is externally covered with the cornea 14 and the sclera 18.

The anterior chamber 13 is the region surrounded by the cornea 14 and the crystalline lens 12. The anterior chamber 13 is circular when viewed from the front. The anterior chamber 13 is filled with aqueous humor.

As illustrated in FIG. 1B, upper and lower portions of the eyeball 10 in the up-down direction are covered with an upper eyelid 19a and a lower eyelid 19b, respectively.

The optical system 20 will now be described.

As illustrated in FIG. 1A, the optical system 20 includes a light radiating unit 20A that radiates light toward the anterior chamber 13 of the eyeball 10, and a light receiving unit 20B that receives the light transmitted through the anterior chamber 13.

The light radiating unit 20A includes a light source unit 21, a collimator lens 22, a deflector 23, and a mirror 27.

The light source unit 21 may be a light source having a narrow wavelength range, such as a laser, or a light source having a wide wavelength range, such as a light emitting diode (LED) or a lamp. The light source unit 21 may include plural lasers, LEDs, or lamps. The light source unit 21 may have plural wavelengths.

The collimator lens 22 collimates diverging light emitted from the light source unit 21 into parallel light (parallel light rays) having a small diameter. Since the anterior chamber 13 surrounded by the cornea 14 and the crystalline lens 12 is small, the light transmitted through the anterior chamber 13 may have a small diameter. Accordingly, the light transmitted through the anterior chamber 13 is beam-shaped.

The collimator lens 22 is not necessary when the light emitted from the light source unit 21 has a small diameter.

In the following description, beam-shaped light may sometimes be referred to as a light beam.

The deflector 23 is a component that changes the direction in which the light travels, and includes, for example, a mirror 231 and a driving device 232 that changes the orientation of a reflective surface of the mirror 231. The mirror 231 may be a galvano mirror or a polygon mirror. The galvano mirror changes the orientation of the reflective surface by rotating the reflective surface around an axis provided on the reflective surface. The polygon mirror changes the orientation of the reflective surface by rotating a polyhedron mirror. The galvano mirror or the polygon mirror changes the orientation of the reflective surface in one direction (one-dimensional direction), so that the direction in which the light travels is changed in one-dimensional direction.

The mirror 231 may instead be composed of a micro electro mechanical system (MEMS). When the reflective surface is arranged so that the orientation thereof may be changed with respect to a point, the orientation of the reflective surface may be changed in one direction and in a direction perpendicular to the one direction. Since the orientation of the reflective surface may be changed in two-dimensional directions, the direction in which the light travels may be changed in two-dimensional directions.

The orientation of the mirror 231 is controlled by the driving device 232. When the mirror 231 is a galvano mirror or a polygon mirror, the driving device 232 includes, for example, a motor and a circuit that controls the motor. When the mirror 231 is a MEMS, the driving device 232 includes, for example, a drive circuit that applies a potential to an electrode integrated with the mirror 231 to control the orientation of the mirror 231 by using an electrostatic force.

The mirror 27 reflects the light from the deflector 23 so that the light passes through the anterior chamber 13. In the first exemplary embodiment, similar to the deflector 23, the mirror 27 is connected to a driving device 28. The mirror 27 is composed of, for example, a galvano mirror, a polygon mirror, or a MEMS. The driving device 28 changes the orientation of the mirror 27 to change the angle at which the incident light is reflected.

The light receiving unit 20B includes a detection unit 29. The detection unit 29 is, for example, a light receiving element such as a silicon diode. The detection unit 29 converts the intensity of the light transmitted through the anterior chamber 13 into an electric signal.

The signal processing unit 30 may be composed of a computer including a central processing unit (CPU), a random access memory (RAM), a read only memory (ROM), a hard disk drive (HDD), and an input/output (I/O) port, and may be operated by software. Alternatively, the signal processing unit 30 may be composed of hardware such as an analog electronic circuit. The signal processing unit 30 receives the electric signal from the detection unit 29 and processes the electric signal to calculate the concentration of the optically active substance contained in the aqueous humor.

The eyeball observation unit 40 is disposed at a position in front of the eyeball 10. While light is radiated toward the anterior chamber 13 of the eyeball 10, the eyeball observation unit 40 observes the optical path relative to the eyeball 10 and outputs image data showing the positional relationship between the eyeball 10 and the optical path. The eyeball observation unit 40 includes an imaging device including, for example, CCD or CMOS devices. The imaging device includes plural light receiving cells (pixels) that are two-dimensionally arranged. Accordingly, the eyeball observation unit 40 outputs image data obtained by the imaging device, the image data showing the positional relationship between the eyeball 10 and the optical path. The controller 60 performs, for example, a binarizing process and an edge detection process on the image data of the eyeball 10 obtained as a result of the observation of the eyeball 10 by the eyeball observation unit 40, and thereby determines the shape of the eyeball 10 and the position of the anterior chamber 13. The controller 60 extracts the pixels corresponding to the wavelength (color) of the light emitted by the light source unit 21 and detects the position at which the eyeball 10 is irradiated with the light.

The line-of-sight guide unit 50 is disposed at a position in front of the eyeball 10, and guides the line of sight of the measurement subject so that the eyeball 10 is in an orientation suitable for the optical measurement. The line-of-sight guide unit 50 includes, for example, a display such as an LCD. A bright or dark spot is shown on the display under the control of the controller 60 so as to guide the line of sight of the measurement subject. At this time, the controller 60 may move the bright or dark spot shown on the display based on the image data output by the eyeball observation unit 40 so that the eyeball 10 is in the orientation suitable for the optical measurement.

In the case where the line-of-sight guide unit 50 includes a display, the calculated concentration of the optically active substance and the image data output by the eyeball observation unit 40 may be shown on the display.

The line-of-sight guide unit 50 may include a sign showing a spot to be looked at by the measurement subject instead of the LCD. The controller 60 may move the sign based on the image data output by the eyeball observation unit 40 so that the eyeball 10 is in the orientation suitable for the optical measurement.

Similar to the signal processing unit 30, the controller 60 may also be composed of a computer including a CPU, a RAM, a ROM, a HDD, and an input/output (I/O) port, and be operated by software. Alternatively, the controller 60 may be composed of hardware, such as an analog electronic circuit. The controller 60 controls the optical system 20, the signal processing unit 30, the eyeball observation unit 40, and the line-of-sight guide unit 50, as described below.

In FIG. 1A, the eyeball observation unit 40 and the line-of-sight guide unit 50 overlap at the position in front of the eyeball 10. Referring to FIG. 1A, the eyeball observation unit 40 may be somewhat shifted from the line-of-sight guide unit 50, which is at the position in front of the eyeball 10, in the up-down direction or in the inward-outward (left-right) direction when viewed from the eyeball 10. In other words, the eyeball observation unit 40 may observe the eyeball 10 from a position shifted diagonally upward or diagonally downward from the position in front of the eyeball 10, or from a position shifted leftward (diagonally inward in this example) or rightward (diagonally outward in this example) from the position directly in front of the eyeball 10. The eyeball 10 may be blocked by the upper eyelid 19a or the lower eyelid 19b when observed from a position shifted diagonally upward or downward, and therefore may be observed from a position shifted leftward or rightward.

When the eyeball observation unit 40 is small and does not serve as an obstacle when the line of sight of the measurement subject is guided, the eyeball observation unit 40 may be disposed between the eyeball 10 and the line-of-sight guide unit 50 so as to overlap the line-of-sight guide unit 50.

### Relationship Between Eyeball 10 and Optical System 20

Next, the relationship between the eyeball 10 and the optical system 20 will be described.

Referring to FIG. 1A, the optical system 20 is set so that the light emitted by the light radiating unit 20A travels along an optical path denoted by α, passes through the anterior chamber 13 of the eyeball 10, and is incident on the light receiving unit 20B. More specifically, as illustrated in FIG. 1A, the optical path α passes through the central region of the anterior chamber 13 in a sectional view of the eyeball 10 along a plane perpendicular to the up-down direction. As illustrated in FIG. 1B, the optical path α also passes through the central region of the anterior chamber 13 in a front view of the eyeball 10.

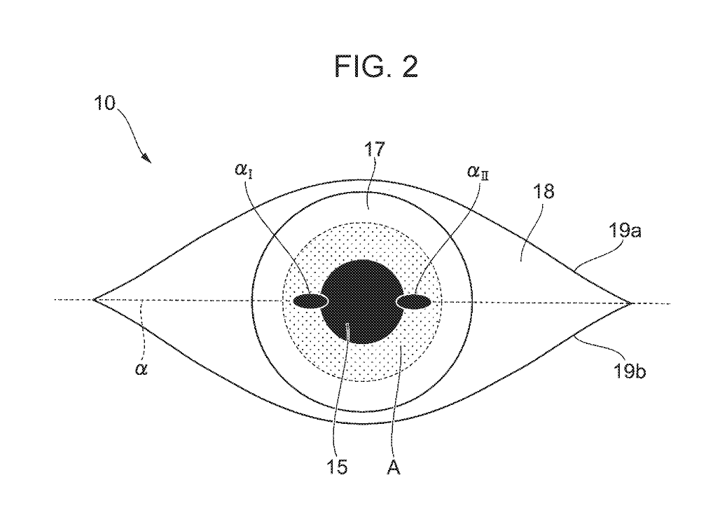

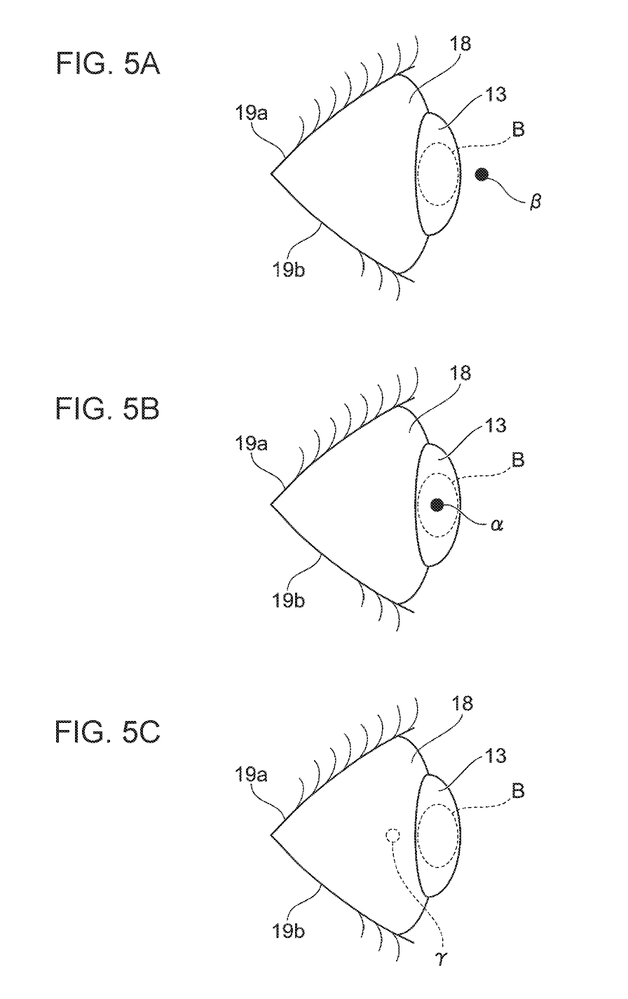

The optical path α is an optical path suitable for the measurement of the concentration of the optically active substance contained in the aqueous humor in the anterior chamber 13. The optical path α has an allowable range in the up-down direction in the front view of the eyeball 10 and in the front-back direction (region denoted by A in FIG. 2 and by B in FIGS. 5A to 5C, which will be described below). The optical path α is any optical path that is within the allowable range and that is suitable for the measurement of the concentration of the optically active substance.

An optical path β illustrated in FIG. 1A is excessively shifted from the optical path α toward the front of the eyeball 10, and passes through a region outside the cornea 14. Thus, the optical path β does not pass through the aqueous humor in the anterior chamber 13. An optical path γ is excessively shifted from the optical path α toward the back of the eyeball 10, and is blocked by the sclera 18. Thus, the optical path γ does not pass through the aqueous humor in the anterior chamber 13.

An optical path δ illustrated in FIG. 1B is excessively shifted from the optical path α toward the top of the eyeball 10, and passes through a region outside the anterior chamber 13. Thus, the optical path δ does not pass through the aqueous humor in the anterior chamber 13. An optical path that is shifted from the optical path δ toward the top of the eyeball 10 may be blocked by the upper eyelid 19a.

An optical path ε is excessively shifted from the optical path α toward the bottom of the eyeball 10, and passes through a region outside the anterior chamber 13. Thus, the optical path ε does not pass through the aqueous humor in the anterior chamber 13. An optical path that is shifted from the optical path ε toward the bottom of the eyeball 10 may be blocked by the lower eyelid 19b.

The terms “optical path α”, “optical path β”, “optical path γ”, “optical path δ”, and “optical path ε” represent the states and positions of the optical path relative to the anterior chamber 13 of the eyeball 10.

The relative positional relationship between the eyeball 10 and the optical system 20 may vary due to, for example, biological vibration of the measurement subject or variation in the shape of the cornea 14 over time. Accordingly, the state of the optical path α may not be maintained. Although the variation in the relative positional relationship may be caused by either a movement of the eyeball 10 relative to the optical system 20 or a movement of the optical system 20 relative to the eyeball 10, it is assumed that the eyeball 10 moves relative to the optical system 20 in the following description for convenience.

When the state of the optical path relative to the eyeball 10 changes from the state of the optical path α to the state of the optical path β or γ, or to the state of the optical path δ or ε, that is, when the optical path is displaced, the optical path may be returned to the state of the optical path α by shifting (moving) or switching the optical path.

For example, referring to FIG. 1A, assume that the eyeball 10 moves backward so that the state of the optical path changes from the state of the optical path α to the state of the optical path β. In this case, a new optical path may be set at the position of the optical path γ. Accordingly, the controller 60 sets an optical path at the position of the optical path γ by controlling the deflector 23 of the light radiating unit 20A so as to switch the incident position on the mirror 27 and controlling the driving device 28 so as to change the orientation of the reflective surface of the mirror 27. In other words, the controller 60 controls the light radiating unit 20A to switch the light incident position on the mirror 27 and change the orientation of the reflective surface of the mirror 27 so that the optical path is moved from the position of the optical path α to the position of the optical path γ. Accordingly, the position at which the eyeball 10 is irradiated with the light is changed and the optical path γ is reset to the optical path α suitable for the measurement of the concentration of the optically active substance contained in the aqueous humor in the anterior chamber 13.

Similarly, assume that the eyeball 10 moves forward so that the state of the optical path changes from the state of the optical path α to the state of the optical path γ. In this case, a new optical path may be set at the position of the optical path β. Accordingly, the controller 60 sets an optical path at the position of the optical path β by controlling the deflector 23 of the light radiating unit 20A so as to switch the incident position on the mirror 27 and controlling the driving device 28 so as to change the orientation of the reflective surface of the mirror 27. In other words, the controller 60 controls the light radiating unit 20A to switch the light incident position on the mirror 27 and change the orientation of the reflective surface of the mirror 27 so that the optical path is moved from the position of the optical path α to the position of the optical path β. Accordingly, the position at which the eyeball 10 is irradiated with the light is changed and the optical path β is reset to the optical path α suitable for the measurement of the concentration of the optically active substance contained in the aqueous humor in the anterior chamber 13.

Also, referring to FIG. 1B, assume that the eyeball 10 moves upward so that the state of the optical path changes from the state of the optical path α to the state of the optical path ε. In this case, a new optical path may be set at the position of the optical path δ. Accordingly, the controller 60 sets an optical path at the position of the optical path δ by controlling the deflector 23 of the light radiating unit 20A so as to switch the incident position on the mirror 27 and controlling the driving device 28 so as to change the orientation of the reflective surface of the mirror 27. In other words, the controller 60 controls the light radiating unit 20A to switch the light incident position on the mirror 27 and change the orientation of the reflective surface of the mirror 27 so that the optical path is moved from the position of the optical path α to the position of the optical path δ. Accordingly, the position at which the eyeball 10 is irradiated with the light is changed and the optical path δ is reset to the optical path α suitable for the measurement of the concentration of the optically active substance contained in the aqueous humor in the anterior chamber 13.

Similarly, assume that the eyeball 10 moves downward so that the state of the optical path changes from the state of the optical path α to the state of the optical path δ. In this case, a new optical path may be set at the position of the optical path ε. Accordingly, the controller 60 sets an optical path at the position of the optical path ε by controlling the deflector 23 of the light radiating unit 20A so as to switch the incident position on the mirror 27 and controlling the driving device 28 so as to change the orientation of the reflective surface of the mirror 27. In other words, the controller 60 controls the light radiating unit 20A to switch the light incident position on the mirror 27 and change the orientation of the reflective surface of the mirror 27 so that the optical path is moved from the position of the optical path α to the position of the optical path ε. Accordingly, the position at which the eyeball 10 is irradiated with the light is changed and the optical path ε is reset to the optical path α suitable for the measurement of the concentration of the optically active substance contained in the aqueous humor in the anterior chamber 13.

The eyeball observation unit 40 observes the optical path relative to the eyeball 10 from a position in front of the eyeball 10, and outputs the image data showing the positional relationship between the eyeball 10 and the optical path. When the optical path is displaced from the optical path α, the controller 60 adjusts the light radiating unit 20A as described above on the basis of the image data output by the eyeball observation unit 40, and resets the optical path to the state of the optical path α suitable for the measurement of the concentration of the optically active substance.

Since the optical path is set on the basis of the image data output by the eyeball observation unit 40, the optical path may be easily set at a target position (predetermined position).

Referring to FIGS. 1A and 1B, the optical path is moved in the front-back direction and/or the up-down direction in a front region of the eyeball 10. In this example, the optical path is translated. This is because the relative positional relationship between the light radiating unit 20A and the light receiving unit 20B of the optical system 20 is maintained. It is not necessary that the optical path be translated.

Although the mirrors 231 and 27 are plane mirrors, they may instead be, for example, concave mirrors, convex mirrors, spherical mirrors, or paraboloidal mirrors.

The incident angles on the mirrors 231 and 27 may be changed not only in the inward-outward direction but also in the up-down direction. When the incident angles are changed in the inward-outward direction, the light incident position on the mirror 27 is switched one-dimensionally in the front-back direction (between the optical paths α, β, and γ). When the incident angles are changed in the up-down direction, the light incident position on the mirror 27 is switched one-dimensionally in the up-down direction (between the optical paths α, δ, and ε). When the incident angles are changed in both the front-back direction and the up-down direction, the light incident position on the mirror 27 is switched two-dimensionally in the front-back direction (between the optical paths α, β, and γ) and the up-down direction (between the optical paths α, δ, and ε).

FIG. 2 is a schematic diagram illustrating the image data output by the eyeball observation unit 40 when the eyeball 10 is observed from the front while the optical path passes through the anterior chamber 13. A laser that emits green light is used as the light source unit 21.

As illustrated in FIG. 2, when the optical path is in the state of the optical path α that passes through the anterior chamber 13, a portion α_{I} of the optical path α near the position at which the light is incident on the anterior chamber 13 and a portion α_{II} of the optical path α near the position at which the light is emitted from the anterior chamber 13 are recognizable. A portion of the optical path α that overlaps the pupil 15 is not recognizable because the pupil 15 is dark.

Thus, the controller 60 is capable of determining the shape of the eyeball 10 and the position of the anterior chamber 13 and detecting the position at which the eyeball 10 is irradiated with the light on the basis of the image data illustrated in FIG. 2.

The shaded area indicates the range in which the optical path α may be positioned (allowable range A).

### Method of Optical Measurement of Eyeball 10

An example of a method of optical measurement of the eyeball 10 (eyeball optical measurement method) will now be described.

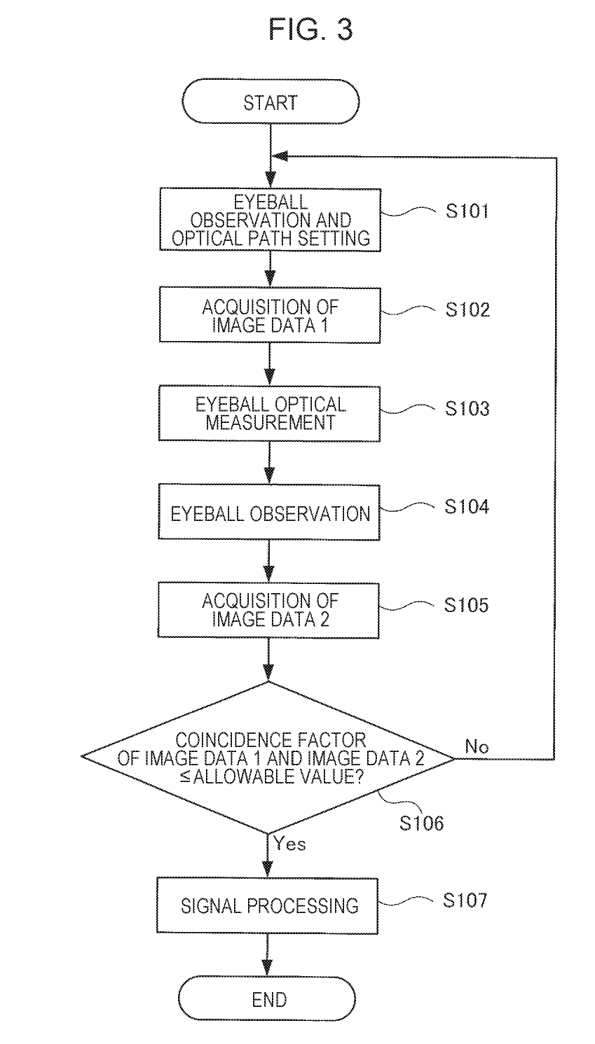

FIG. 3 is a flowchart of an example of a method of optical measurement of the eyeball 10.

Here, it is assumed that the optical measurement apparatus 1 has already been placed on the eyeball 10 of the measurement subject.

Also, it is assumed that the procedure of the flowchart is carried out by the controller 60.

First, the eyeball observation unit 40 observes the optical path relative to the eyeball 10, and the acquired image data is used to determine the shape of the eyeball 10 and the position of the anterior chamber 13 and detect the position at which the eyeball 10 is irradiated with the light. Then, it is determined whether the optical path is outside the allowable range A of the optical path α (see FIG. 2). When it is determined that the optical path is outside the allowable range A of the optical path α, the light radiating unit 20A is controlled so that the position at which the eyeball 10 is irradiated with the light is changed to a position where the optical path is within the allowable range A of the optical path α. More specifically, the deflector 23 is controlled so as to change the light incident position on the mirror 27, and the driving device 28 is controlled so as to change the orientation of the reflective surface of the mirror 27. Thus, the optical path is set to the optical path α (eyeball observation and optical path setting). This step is step 101, which is denoted by S101 in FIG. 3 (this also applies to the following steps).

Then, the image data in which the optical path is set to the optical path α (image data 1) is acquired and stored in a memory (acquisition of image data 1) (step 102).

Image data 1 is an example of first image data, and steps 101 and 102 are an example of a step of observing the optical path relative to the eyeball and acquiring first image data representing the positional relationship between the eyeball and the optical path.

Then, the optical measurement of the eyeball 10 is performed (eyeball optical measurement) (step 103).

Step 103 is an example of a step of performing the optical measurement on the anterior chamber of the eyeball.

After the optical measurement of the eyeball 10, the eyeball observation unit 40 observes the optical path relative to the eyeball 10 again, and the acquired image data is used to determine the shape of the eyeball 10 and the position of the anterior chamber 13 and detect the position at which the eyeball 10 is irradiated with the light (eyeball observation) (step 104).

Then, the image data of the observed eyeball 10 (image data 2) is acquired and stored in a memory (acquisition of image data 2) (step 105).

Image data 2 is an example of second image data, and steps 104 and 105 are an example of a step of observing the optical path relative to the eyeball and acquiring second image data representing the positional relationship between the eyeball and the optical path.

Next, it is determined whether or not a coincidence factor of image data 1 and image data 2 is less than or equal to an allowable value (step 106). The allowable value is, for example, an amount of displacement (distance) between the optical path in image data 2 and the optical path in image data 1. The expression “the coincidence factor is less than or equal to the allowable value” means that, for example, the amount of displacement (distance) is within a predetermined range. The predetermined range is set on the basis of, for example, an error in the concentration of the optically active substance caused when the length of the optical path in the anterior chamber 13 varies due to a displacement of the optical path.

When the result of the determination is Yes in step 106, that is, when the coincidence factor of image data 1 and image data 2 is less than or equal to the predetermined allowable value, the signal processing unit 30 processes the signal obtained by the optical measurement of the eyeball 10 in step 103 to calculate the concentration of the optically active substance (step 107).

Steps 106 and 107 are an example of a step of processing the signal obtained by the optical measurement when the coincidence factor of the first image data and the second image data is less than or equal to the predetermined allowable value.

When the result of the determination is No in step 106, that is, when the coincidence factor of image data 1 and image data 2 is greater than the predetermined allowable value, the process returns to step 101. When it is determined that the optical path is outside the allowable range A of the optical path α on the basis of image data 2, the light radiating unit 20A is readjusted so that the optical path is within the allowable range A of the optical path α. When it is determined that the optical path is within the allowable range A of the optical path α on the basis of image data 2, the process may proceed to step 102.

Image data 1 is overwritten in step 102, and image data 2 is overwritten in step 105.

In the flowchart of FIG. 3, the image data of the eyeball 10 before the optical measurement of the eyeball 10 and that after the optical measurement of the eyeball 10 (image data 1 and image data 2) are compared. When the displacement of the optical path is less than or equal to the allowable value, signal processing is performed to calculate the concentration of the optically active substance. As a result, the reliability of the position of the optical path during the optical measurement of the anterior chamber 13 is increased, and the accuracy of the calculated concentration of the optically active substance is increased accordingly.

When the accuracy of the concentration of the optically active substance is sufficiently high, step 104 to step 106 in FIG. 3 may be omitted.

As described above, in the optical measurement apparatus 1 according to the first exemplary embodiment, even when the relative positional relationship between the eyeball 10 and the optical system 20 varies due to, for example, biological vibration of the measurement subject or variation in the shape of the cornea 14 over time, the optical path is reset to the state of the optical path α, that is, so as to pass through the anterior chamber 13 based on the image data of the eyeball 10 obtained by the eyeball observation unit 40.

Whether or not the optical path is displaced from the optical path α may be determined based on the signal from the detection unit 29. For example, referring to FIGS. 1A and 1B, the signal from the detection unit 29 increases when the optical path is shifted from the optical path α to the optical path β toward the front of the eyeball 10. Also, the signal from the detection unit 29 decreases when the optical path is shifted from the optical path α to the optical path γ toward the back of the eyeball 10. However, the optical path may be more accurately detected when the eyeball observation unit 40 observes the optical path relative to the eyeball 10 than when the signal from the detection unit 29 is used.

### Second Exemplary Embodiment

In the optical measurement apparatus 1 according to the first exemplary embodiment, the eyeball observation unit 40 observes the optical path relative to the eyeball 10 from a position in front of the eyeball 10. When the eyeball 10 is observed from the front, a displacement of the optical path in the up-down direction is easily detectable, but a displacement of the optical path in the front-back direction is not easily detectable. Also when the eyeball 10 is observed diagonally from a position that is shifted leftward or rightward from the position in front of the eyeball 10, a displacement of the optical path in the front-back direction is not easily detectable.

Accordingly, in an optical measurement apparatus 1 of a second exemplary embodiment, the optical path is observed relative to the eyeball 10 by observing the eyeball 10 from a position that is further shifted leftward or rightward from the position in front of the eyeball 10, that is, by observing the eyeball 10 sideways from a position on one side of the eyeball 10 or from a position close to the position on one side of the eyeball 10.

The position on one side of the eyeball 10 or the position close to the position on one side of the eyeball 10 described in the second and succeeding exemplary embodiments is also referred to as a position shifted leftward or rightward from the position in front of the eyeball 10.

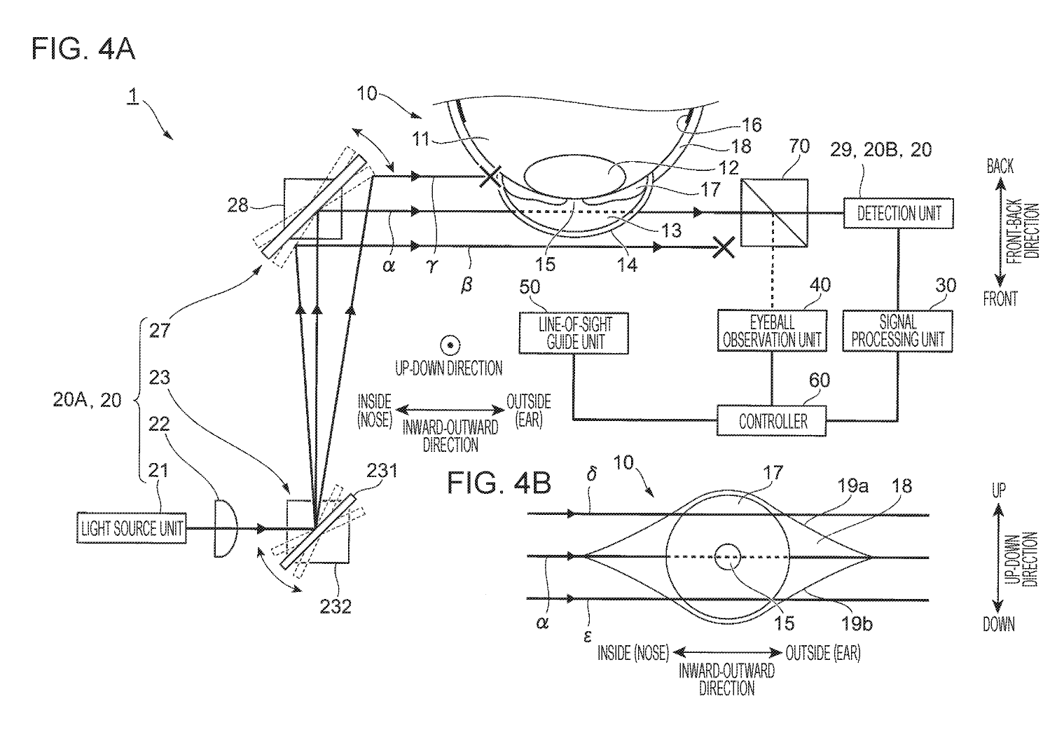

FIGS. 4A and 4B illustrate an exemplary structure of the eyeball optical measurement apparatus 1 according to the second exemplary embodiment. FIG. 4A shows a top view of the eyeball 10 (sectional view along a plane perpendicular to the up-down direction), and FIG. 4B shows a front view of the eyeball 10.

The optical measurement apparatus 1 according to the second exemplary embodiment includes a beam splitter 70 disposed on the optical path between the eyeball 10 and the detection unit 29 in the optical measurement apparatus 1 according to the first exemplary embodiment illustrated in FIG. 1. The eyeball observation unit 40 is arranged to observe the eyeball 10 via a reflective surface of the beam splitter 70 from a position shifted rightward (outward) from the position in front of the eyeball 10. Other structures are similar to those in the optical measurement apparatus 1 according to the first exemplary embodiment and therefore denoted by the same reference numerals, and description thereof is omitted.

When the eyeball 10 is a right eyeball, the eyeball observation unit 40 is arranged to observe the eyeball 10 via the reflective surface of the beam splitter 70 from a position shifted leftward (outward) from the position in front of the eyeball 10.

The beam splitter 70 splits a single light beam into plural light beams (two light beams in this example). The beam splitter 70 may be, for example, a cube-shaped beam splitter obtained by joining diagonal surfaces of two right-angle prisms with an optical thin film interposed therebetween, or a plate-shaped half mirror having an optical thin film or a thin metal film provided on one surface thereof.

The eyeball observation unit 40 observes the optical path relative to the eyeball 10 via the reflective surface of the beam splitter 70.

FIGS. 5A to 5C illustrate image data obtained by the eyeball observation unit 40. FIG. 5A illustrates the optical path β that is in front of the anterior chamber 13 of the eyeball 10. FIG. 5B illustrates the optical path α that passes through the anterior chamber 13. FIG. 5C illustrates the optical path γ that is behind the anterior chamber 13 of the eyeball 10. The optical path α, which passes through the anterior chamber 13, has an allowable range in the side view image of the eyeball 10 (region denoted by B in FIG. 5). Any optical path within the allowable range is referred to as the optical path α suitable for the measurement of the concentration of the optically active substance.

As illustrated in FIG. 5A, when the optical path is in front of the anterior chamber 13 of the eyeball 10 and is at the position of the optical path β, the optical path β is directly observed in front of the eyeball 10.

As illustrated in FIG. 5B, when the optical path is at the position of the optical path α that passes through the anterior chamber 13 of the eyeball 10, the light transmitted through the anterior chamber 13 is observed.

As illustrated in FIG. 5C, when the optical path is behind the anterior chamber 13 of the eyeball 10 and is at the position of the optical path γ, light is blocked by the sclera 18 of the eyeball 10 and cannot be observed.

Although not described herein, this also applies to displacements in the up-down direction.

Thus, displacements of the optical path in the front-back direction and the up-down direction may be easily detected on the basis of the image data obtained by observing the eyeball 10 from a position shifted leftward or rightward from the position in front of the eyeball 10.

Accordingly, the controller 60 may control the light radiating unit 20A (deflector 23 and mirror 27) on the basis of the image data so as to change the position at which the eyeball 10 is irradiated with the light to a position where the optical path is at the position of the optical path α.

The optical measurement apparatus 1 according to the second exemplary embodiment may use the method of optical measurement of the eyeball 10 according to the first exemplary embodiment described with reference to FIG. 3.

### Third Exemplary Embodiment

In the optical measurement apparatus 1 according to the second exemplary embodiment, the beam splitter 70 is disposed on the optical path between the eyeball 10 and the detection unit 29. When the beam splitter 70 is disposed on the optical path, the amount of light incident on the detection unit 29 decreases due to the beam splitter 70.

Accordingly, in an optical measurement apparatus 1 of a third exemplary embodiment, a movable mirror 80 is provided in place of the beam splitter 70 included in the optical measurement apparatus 1 according to the second exemplary embodiment.

The movable mirror 80 is an example of an optical-path switching unit.

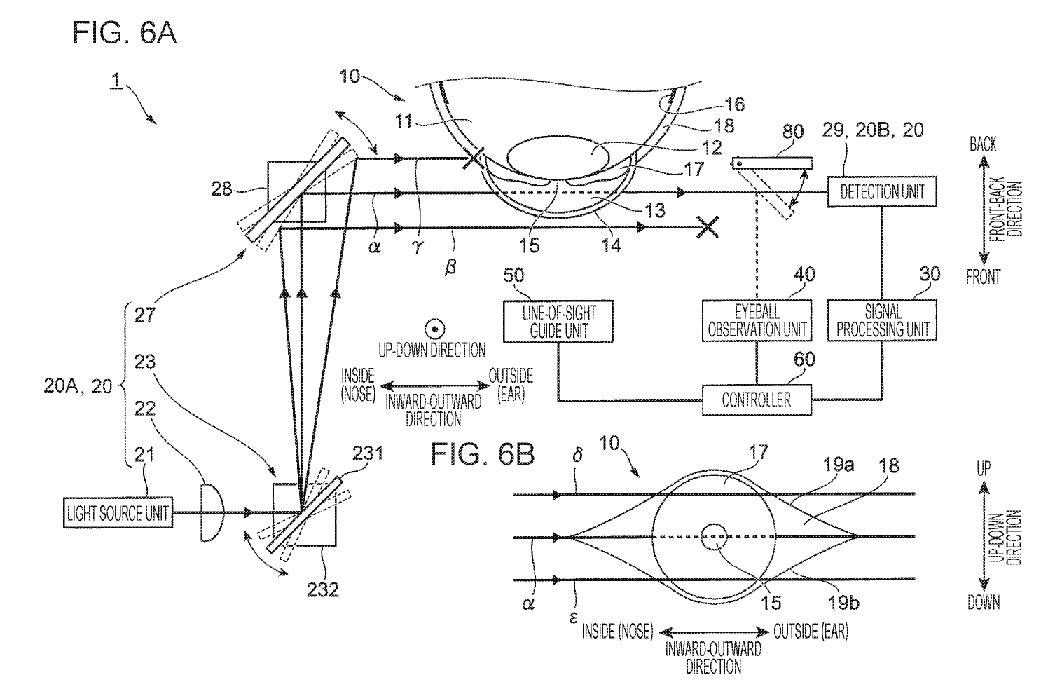

FIGS. 6A and 6B illustrate an exemplary structure of the eyeball optical measurement apparatus 1 according to the third exemplary embodiment. FIG. 6A shows a top view of the eyeball 10 (sectional view along a plane perpendicular to the up-down direction), and FIG. 6B shows a front view of the eyeball 10.

In the optical measurement apparatus 1 according to the third exemplary embodiment, the movable mirror 80 for switching the optical path is disposed on the optical path between the eyeball 10 and the detection unit 29 in the optical measurement apparatus 1 according to the first exemplary embodiment illustrated in FIG. 1. The movable mirror 80 is a mirror having a movable reflective surface, and switches the optical path so that light that travels in a certain direction travels in another direction.

When the eyeball observation unit 40 observes the optical path relative to the eyeball 10, the reflective surface of the movable mirror 80 is placed on the optical path. Accordingly, the optical path is switched so that the light transmitted through the anterior chamber 13 of the eyeball 10 travels toward the eyeball observation unit 40.

Then, the optical path is set to the state of the optical path α that is suitable for the measurement of the concentration of the optically active substance. After the optical path is set, the reflective surface of the movable mirror 80 is removed (flipped up) from the optical path. Accordingly, the optical path is switched so that the light transmitted through the anterior chamber 13 of the eyeball 10 is incident on the detection unit 29. Thus, the light transmitted through the anterior chamber 13 is directly incident on the detection unit 29.

When the eyeball observation unit 40 observes the optical path relative to the eyeball 10, the reflective surface of the movable mirror 80 causes the optical path to extend toward the eyeball observation unit 40. Therefore, the position of the reflective surface on the optical path may be fixed. Accordingly, when the reflective surface of the movable mirror 80 is placed on the optical path, the reflective surface may be pressed against a guide so that the direction in which the light is reflected by the reflective surface is fixed.

When the reflective surface of the movable mirror 80 is removed (flipped up) from the optical path, it is not necessary to fix the position of the reflective surface as long as the reflective surface of the movable mirror 80 does not block the optical path.

Although the movable mirror 80 is removed from the optical path by flipping up the movable mirror 80, the movable mirror 80 may instead be removed from the optical path by sliding the movable mirror 80.

According to the optical measurement apparatus 1 of the third exemplary embodiment, unlike the optical measurement apparatus 1 of the second exemplary embodiment, the amount of light incident on the detection unit 29 is not reduced.

The optical measurement apparatus 1 according to the third exemplary embodiment may use the method of optical measurement of the eyeball 10 according to the first exemplary embodiment described with reference to FIG. 3.

### Fourth Exemplary Embodiment

In the optical measurement apparatuses 1 according to the first to third exemplary embodiments, the optical path toward the eyeball 10 is changed by using the mirror 231 of the deflector 23 and the mirror 27 included in the light radiating unit 20A.

In a fourth exemplary embodiment, the light incident angle on the mirror 27 is fixed, and the optical path toward the eyeball 10 is changed by using the mirror 231 of the deflector 23.

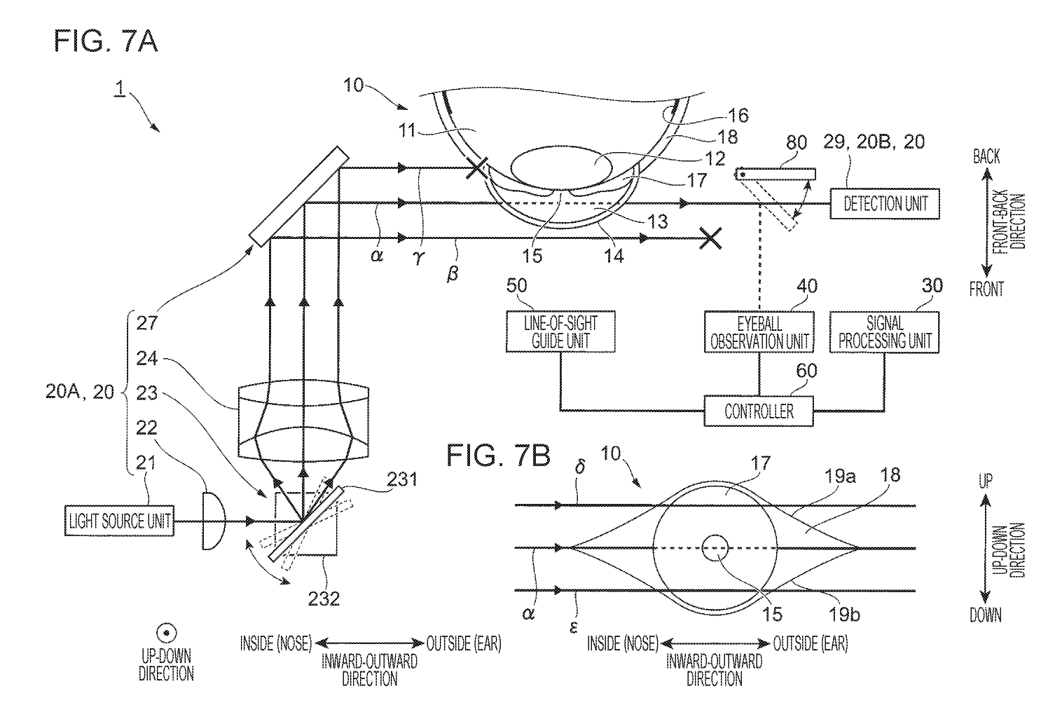

FIGS. 7A and 7B illustrate an exemplary structure of an eyeball optical measurement apparatus 1 according to the fourth exemplary embodiment. FIG. 7A shows a top view of the eyeball 10 (sectional view along a plane perpendicular to the up-down direction), and FIG. 7B shows a front view of the eyeball 10.

In the fourth exemplary embodiment, the movable mirror 80 according to the third exemplary embodiment is used. Therefore, components similar to those of the optical measurement apparatus 1 according to the third exemplary embodiment are denoted by the same reference numerals, and description thereof is thus omitted.

The eyeball observation unit 40 may observe the optical path relative to the eyeball 10 from a position in front of the eyeball 10 as described in the first exemplary embodiment. Alternatively, the beam splitter 70 may be used in place of the movable mirror 80 to observe the optical path relative to the eyeball 10 from a position shifted in the left-right direction from the position in front of the eyeball 10 as described in the second exemplary embodiment.

In the eyeball optical measurement apparatus 1 according to the fourth exemplary embodiment, a telecentric optical system 24 including a telecentric fθ lens is disposed between the deflector 23 and the mirror 27. The mirror 27 is not provided with the driving device 28 according to the first exemplary embodiment.

The telecentric fθ lens collects light incident thereon so that the light becomes perpendicular to a flat surface. As illustrated in FIG. 7A, even when light beams are reflected by the mirror 231 of the deflector 23 so as to be incident on the telecentric optical system 24 at an angle, light beams emitted from the telecentric optical system 24 are parallel to each other.

Accordingly, even when the light incident angle of the mirror 27 (orientation of the mirror 27) is fixed, the light incident on the eyeball 10 may be translated by changing the light incident position on the mirror 27. In other words, the optical path may be moved in the front-back direction and/or the up-down direction of the eyeball 10.

Thus, the light incident position on the mirror 27 may be changed by controlling the reflection angle of the mirror 231 of the deflector 23. In other words, the light incident position on the mirror 27 may be easily controlled.

Since the mirror 27 is disposed near the eyeball 10, in the optical measurement apparatuses 1 according to the first to third exemplary embodiments, dynamic force is applied to the measurement subject when the mirror 27 is moved (rotated) to change the incident angle on the mirror 27. In contrast, in the optical measurement apparatus 1 according to the fourth exemplary embodiment, no dynamic force is applied to the measurement subject since the incident angle of the mirror 27 is fixed.

The process of changing the incident position on the mirror 27 is similar to that in the first exemplary embodiment except that the incident angle on the mirror 27 (orientation of the mirror 27) is fixed, and description thereof is thus omitted.

The optical measurement apparatus 1 according to the fourth exemplary embodiment may use the method of optical measurement of the eyeball 10 according to the first exemplary embodiment described with reference to FIG. 3.

### Fifth Exemplary Embodiment

In the first to fourth exemplary embodiments, the concentration of the optically active substance contained in the aqueous humor is measured on the basis of a change in the intensity of light transmitted through the aqueous humor in the anterior chamber 13.

In a fifth exemplary embodiment, the concentration of the optically active substance, such as glucose, contained in the aqueous humor is measured by utilizing optical activity (optical rotation).

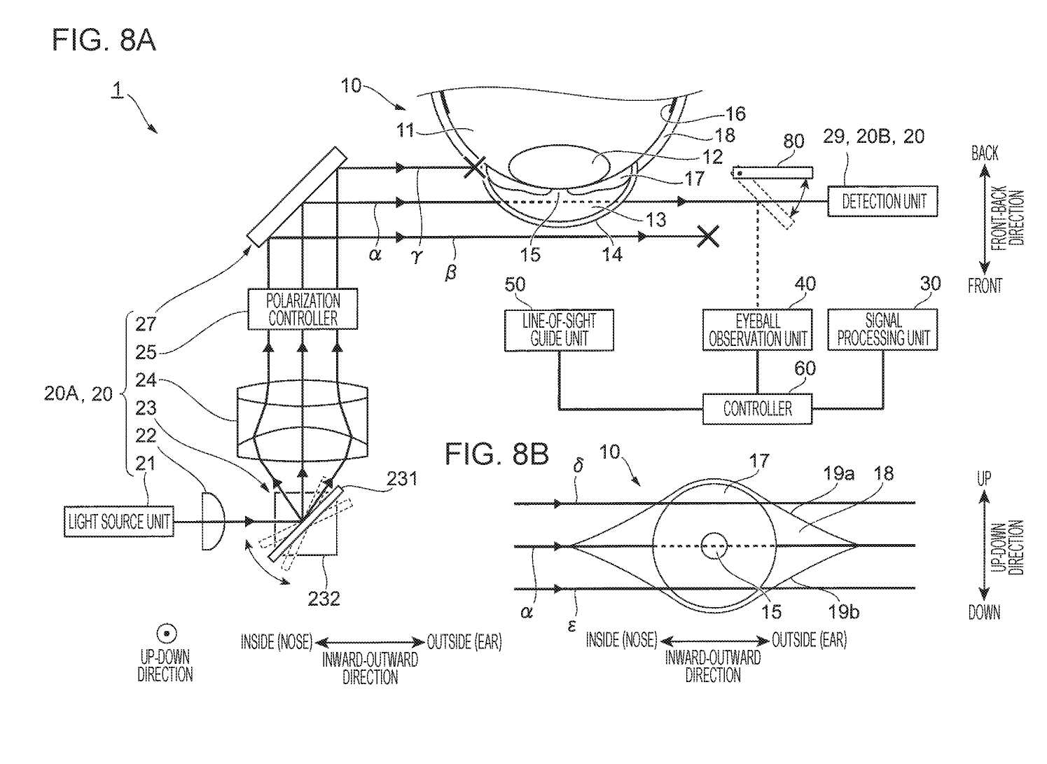

FIGS. 8A and 8B illustrate an exemplary structure of an eyeball optical measurement apparatus 1 according to the fifth exemplary embodiment. FIG. 8A shows a top view of an eyeball 10 (sectional view along a plane perpendicular to the up-down direction), and FIG. 8B shows a front view of the eyeball 10. Components similar to those of the optical measurement apparatus 1 according to the fourth exemplary embodiment are denoted by the same reference numerals, and description thereof is thus omitted.

The optical measurement apparatus 1 according to the fifth exemplary embodiment is the optical measurement apparatus 1 according to the fourth exemplary embodiment including a polarization controller 25.

The polarization controller 25 includes a polarizer and a wave plate. Predetermined polarized light (for example, linearly polarized light, elliptically polarized light, or circularly polarized light) is extracted from the light emitted by the light source unit 21.

When the mirror 27 reflects light, the reflectances of a component (P) parallel to the incident surface and a component (S) perpendicular to the incident surface depend on the refractive index of the mirror 27 and the incident angle on the mirror 27. Therefore, when polarized light is incident on the mirror 27, the state of polarization of the reflected light may vary depending on the incident angle. For example, when the incident light is linearly polarized, the reflected light is also linearly polarized when the incident angle is a certain angle, and is elliptically polarized when the incident angle is another angle.

Therefore, the incident angle on the mirror 27 may be fixed.

Accordingly, in the optical measurement apparatus 1 of the fifth exemplary embodiment, similar to the fourth exemplary embodiment, the telecentric optical system 24 including a telecentric fθ lens is used so that it is not necessary to consider a change in the state of polarization due to a change in the incident angle on the mirror 27.

The state of polarization also changes when polarized light passes through a lens. Therefore, the polarization controller 25 is disposed behind the telecentric fθ lens of the telecentric optical system 24, that is, between the telecentric fθ lens and the mirror 27.

The detection unit 29 includes an analyzer for detecting an angle of rotation, as described below.

In the optical measurement apparatus 1 according to the fifth exemplary embodiment, similar to the optical measurement apparatus 1 according to the fourth exemplary embodiment, the movable mirror 80 is used to switch the optical path between the optical path toward the eyeball observation unit 40 and the optical path toward the detection unit 29. Therefore, the light incident on the detection unit 29 is not influenced by a change in the state of polarization due to the movable mirror 80.

A change in the state of polarization of the reflected light may be calculated if the refractive index of the mirror 27, the state of polarization of the incident light (orientation of the plane of polarization and whether the light is linearly or elliptically polarized), and the incident angle are known. Therefore, also in the optical measurement apparatuses 1 according to the first and third exemplary embodiments, the concentration of the optically active substance may be measured by utilizing optical activity when the polarization controller 25 is additionally provided.

Also in the optical measurement apparatus 1 according to the second exemplary embodiment, similar to a change in the state of polarization due to the mirror 27, a change in the state of polarization due to the beam splitter 70 may be calculated. Therefore, also in the optical measurement apparatus 1 according to the second exemplary embodiment, the concentration of the optically active substance may be measured by utilizing optical activity when the polarization controller 25 is additionally provided.

Although the concentration of the optically active substance is measured by utilizing optical activity (optical rotation), the process of changing the light incident position on the mirror 27 is similar to that in the fourth exemplary embodiment, and description thereof is thus omitted.

### Calculation of Concentration of Optically Active Substance

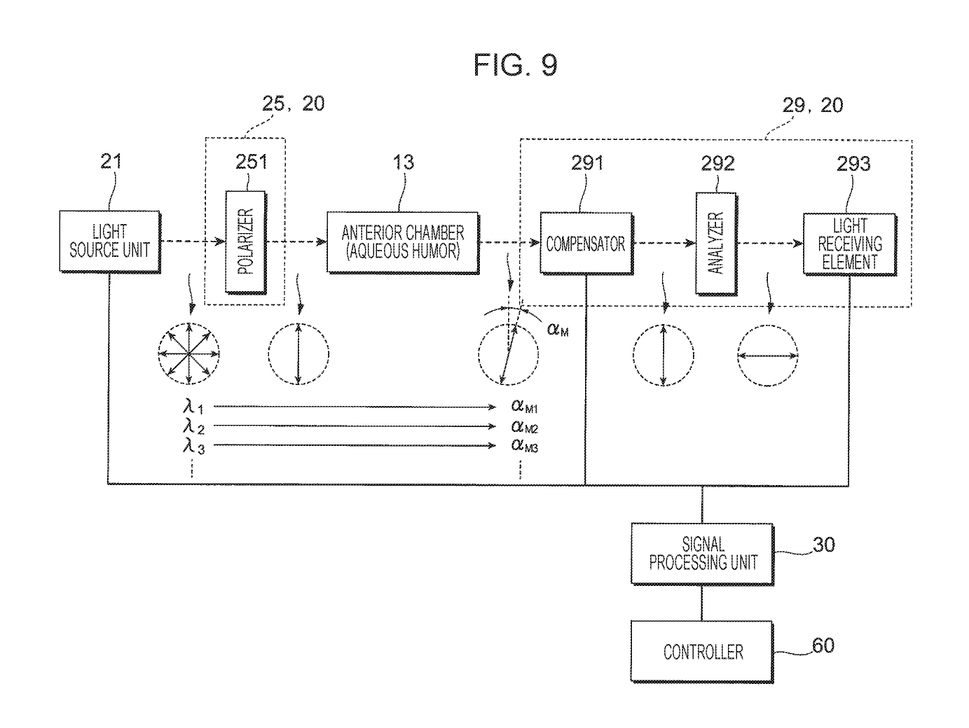

FIG. 9 illustrates a method by which optical measurement apparatus 1 measures an angle of rotation of the plane of polarization (optical rotation) due to an optically active substance contained in the aqueous humor in the anterior chamber 13. Here, to facilitate description, it is assumed that the optical path is not bent, and the telecentric optical system 24 and the mirror 27 are omitted.

The polarization controller 25 included in the optical system 20 includes a polarizer 251, and the detection unit 29 included in the optical system 20 includes a compensator 291, an analyzer 292, and a light receiving element 293.

Referring to FIG. 9, the arrows in circles shown between the light source unit 21, the polarizer 251 of the polarization controller 25, the anterior chamber 13, and the compensator 291, the analyzer 292, and the light receiving element 293 of the detection unit 29 indicate the state of polarization when viewed in the direction in which the light travels. In this specification, the plane of polarization of linearly polarized light is the plane along which the electric field of the linearly polarized light vibrates.

The optical system 20 may include other elements (for example, optical components).

The polarizer 251 is, for example, a Nicol prism, a total internal reflection Glan-Thompson prism, a Glan-Taylor prism, or a Glan-laser prism, and transmits linearly polarized light having a predetermined plane of polarization when light is incident thereon.

The compensator 291 is, for example, a magneto-optical element such as a Faraday element that uses garnet or the like, and rotates a plane of polarization of linearly polarized light by using a magnetic field.

The analyzer 292 is a component similar to the polarizer 251, and transmits linearly polarized light having a predetermined plane of polarization.

The light receiving element 293 is, for example, a silicon diode, and outputs an output signal that corresponds to the intensity of light.

The light source unit 21 emits light having various planes of polarization. The polarizer 251 transmits linearly polarized light having a predetermined plane of polarization. In FIG. 9, for example, the polarizer 251 transmits linearly polarized light having a plane of polarization parallel to the plane of FIG. 9.

The plane of polarization of the linearly polarized light transmitted through the polarizer 251 is rotated due to optically active substances contained in the aqueous humor in the anterior chamber 13. In FIG. 9, the plane of polarization is rotated by an angle of α_{M} (optical rotation is α_{M}).

Next, the plane of polarization that has been rotated by the optically active substances contained in the aqueous humor in the anterior chamber 13 is returned by the compensator 291. When the compensator 291 is a magneto-optical element such as a Faraday element, the plane of polarization of the light transmitted through the compensator 291 is rotated by applying a magnetic field to the compensator 291.

The light receiving element 293 receives the linearly polarized light transmitted through the analyzer 292, and converts the received light into an output signal that corresponds to the intensity of the received light.

An example of a method for measuring the optical rotation α_{M} by using the optical system 20 will now be described.

First, in the state in which the light emitted from the light source unit 21 does not pass through the anterior chamber 13, the compensator 291 and the analyzer 292 are set so that the output signal from the light receiving element 293 is minimized by the optical system 20 including the light source unit 21, the polarizer 251, the compensator 291, the analyzer 292, and the light receiving element 293. In the example illustrated in FIG. 9, when the light does not pass through the anterior chamber 13, the plane of polarization of the linearly polarized light transmitted through the polarizer 251 is perpendicular to the plane of polarization of light transmitted through the analyzer 292.

Next, the light is caused to pass through the anterior chamber 13. In this state, the plane of polarization is rotated due to the optically active substances contained in the aqueous humor in the anterior chamber 13. Therefore, the output signal from the light receiving element 293 changes from the minimum value. Accordingly, a magnetic field is applied to the compensator 291 to rotate the plane of polarization so that the output signal from the light receiving element 293 is minimized. In other words, the plane of polarization of the light emitted from the compensator 291 is set so as to be perpendicular to the plane of polarization of the light transmitted through the analyzer 292.

The angle by which the plane of polarization is rotated by the compensator 291 corresponds to the optical rotation α_{M} caused by the optically active substances contained in the aqueous humor. The relationship between the magnitude of the magnetic field applied to the compensator 291 and the angle by which the plane of polarization is rotated is known in advance. Accordingly, the optical rotation α_{M} may be determined on the basis of the magnitude of the magnetic field applied to the compensator 291.

More specifically, light beams having different wavelengths λ (λ₁, λ₂, λ₃, . . . ) are emitted by the light source unit 21 so as to be incident on the aqueous humor in the anterior chamber 13, and the optical rotations α_{M} (α_{M1}, α_{M2}, α_{M3}, . . . ) are determined for the respective light beams. The signal processing unit 30 receives the combinations of the wavelengths λ and optical rotations α_{M}, and calculates the concentration of the target optically active substance.

As described above, the aqueous humor contains plural optically active substances. Therefore, the measured optical rotation α_{M} is the sum of the optical rotations α_{M} caused by the respective optically active substances. Accordingly, it is necessary to calculate the concentration of the target optically active substance (glucose in this example) from the measured optical rotation α_{M}. The concentration of the target optically active substance may be calculated by a known method, and description thereof will thus be omitted herein.

Referring to FIG. 9, the plane of polarization of the light transmitted through the polarizer 251 is parallel to the plane of FIG. 9, and the plane of polarization of the light before transmission through the analyzer 292 is also parallel to the plane of FIG. 9. However, in the case where the plane of polarization of the light that is emitted from the light source unit 21 and that does not through the anterior chamber 13 is rotated by the compensator 291, the plane of polarization of the light before transmission through the analyzer 292 may be at an angle relative to the plane parallel to the plane of FIG. 9. The orientation of the plane of polarization is not particularly limited as long as the compensator 291 and the analyzer 292 are set so as to minimize the output signal from the light receiving element 293 when the light does not pass through the aqueous humor in the anterior chamber 13.

Although the compensator 291 is used to determine the optical rotation α_{M} in this example, the optical rotation α_{M} may instead be determined by using a component other than the compensator 291. In addition, although the crossed-polarizers method, which is the most basic method for measuring the rotation angle of a plane of polarization (optical rotation α_{M}), is used in this example (with the use of the compensator 291), any other measurement method, such as the rotating analyzer method, Faraday modulation method, or optical delay modulation method, may instead be used.

The optical measurement apparatus 1 according to the fifth exemplary embodiment may use the method of optical measurement of the eyeball 10 according to the first exemplary embodiment described with reference to FIG. 3.

### Sixth Exemplary Embodiment

In the optical measurement apparatus 1 according to the fifth exemplary embodiment, the incident angle on the mirror 27 is fixed by using the telecentric optical system 24 including the telecentric fθ lens.

In an optical measurement apparatus 1 according to the sixth exemplary embodiment, the optical path is switched by moving the mirror 231 of the deflector 23 instead of using the telecentric optical system 24.

In the sixth exemplary embodiment, the polarization controller 25 is provided to measure the concentration of the optically active substance, such as glucose, by utilizing optical activity (optical rotation).

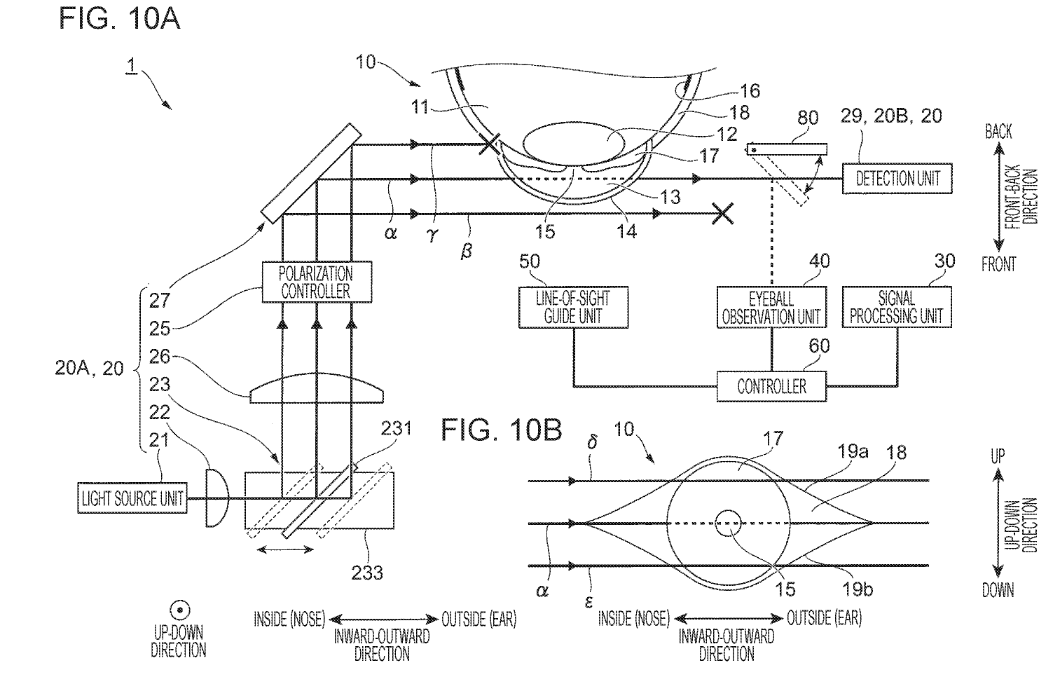

FIGS. 10A and 10B illustrate an exemplary structure of the eyeball optical measurement apparatus 1 according to the sixth exemplary embodiment. FIG. 10A shows a top view of the eyeball 10 (sectional view along a plane perpendicular to the up-down direction), and FIG. 10B shows a front view of the eyeball 10. In the following description, elements similar to those of the optical measurement apparatus 1 according to the fifth exemplary embodiment illustrated in FIGS. 8A and 8B are denoted by the same reference numerals, and description thereof will be omitted. Differences from the optical measurement apparatus 1 according to the fifth exemplary embodiment will now be described.

In the optical measurement apparatus 1 according to the sixth exemplary embodiment, a converging lens 26 is provided in place of the telecentric optical system 24. The deflector 23 includes the mirror 231 and a linear stage 233 that moves in one direction while carrying the mirror 231.

The linear stage 233 moves the reflective surface of the mirror 231 in the direction of the optical path (direction in which the light travels). Accordingly, the light incident position on the mirror 27 is changed. Thus, the optical path is set to the state of the optical path α suitable for the measurement of the concentration of the optically active substance contained in the aqueous humor in the anterior chamber 13. In other words, the optical path is set so as to pass through the anterior chamber 13.

In the sixth exemplary embodiment, the direction in which the light incident position on the mirror 27 is moved is limited by the direction in which the linear stage 233 is moved. In other words, the light incident position on the mirror 27 is changed in a one-dimensional direction. For example, in FIG. 10A, the optical path is moved only in the front-back direction of the face.

Therefore, when the optical path is to be moved in the up-down direction of the face as illustrated in FIG. 10B, the light source unit 21 and the collimator lens 22 are arranged in a direction perpendicular to the plane of FIG. 10A, and the linear stage 233 is moved in the direction perpendicular to the plane of FIG. 10A. In addition, the mirror 231 on the linear stage 233 is oriented so that the light emitted from the light source unit 21 and transmitted through the collimator lens 22 is reflected toward the mirror 27.

Instead of using the linear stage 233, the front surface of the mirror 231 may be moved by using a piezoelectric element attached to the back surface of the mirror 231. In such a case, the linear stage 233 may be replaced by a driving device that drives the piezoelectric element.

The eyeball observation unit 40 may observe the optical path relative to the eyeball 10 from a position in front of the eyeball 10 as described in the first exemplary embodiment. Alternatively, the beam splitter 70 may be used in place of the movable mirror 80 to observe the optical path relative to the eyeball 10 from a position shifted in the left-right direction from the position in front of the eyeball 10 as described in the second exemplary embodiment.

The optical measurement apparatus 1 according to the sixth exemplary embodiment may use the method of optical measurement of the eyeball 10 according to the first exemplary embodiment described with reference to FIG. 3.

### Seventh Exemplary Embodiment

In an eyeball optical measurement apparatus 1 according to a seventh exemplary embodiment, the periphery of the anterior chamber 13 of the eyeball 10 is immersed in liquid. This state may be referred to as an immersed state.

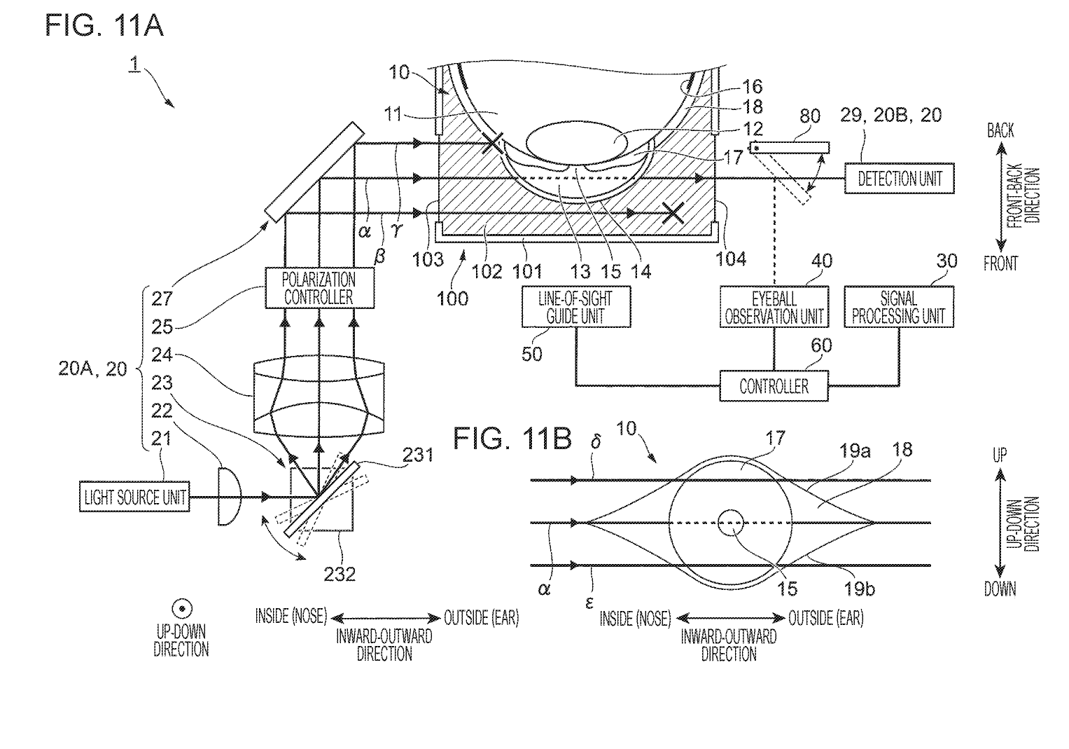

FIGS. 11A and 11B illustrate an exemplary structure of the eyeball optical measurement apparatus 1 according to the seventh exemplary embodiment. FIG. 11A shows a top view of the eyeball 10 (sectional view along a plane perpendicular to the up-down direction), and FIG. 11B shows a front view of the eyeball 10. The structure of the optical measurement apparatus 1 is similar to that in the fifth exemplary embodiment illustrated in FIGS. 8A and 8B except for an immersion unit 100 described below. Therefore, similar elements are denoted by the same reference numerals, and description thereof will be omitted. Differences from the fifth exemplary embodiment will now be described.

The immersion unit 100 includes a container 101 and liquid 102 that fills the container 101. The container 101 of the immersion unit 100 is pressed against the surface of a portion of the face around the eyeball 10, so that the periphery the anterior chamber 13 of the eyeball 10 is immersed in the liquid 102. The liquid 102 may have a refractive index that is close to that of the aqueous humor. The liquid 102 may be, for example, water or saline.

The container 101 of the immersion unit 100 has an entrance window 103 and an exit window 104, which allow the light to pass therethrough, at positions corresponding to the optical path so that the light may be transversely transmitted through the anterior chamber 13. The entrance window 103 is arranged so that the light reflected by the mirror 27 enters through the entrance window 103 in a direction perpendicular thereto, and the exit window 104 is arranged so that the light transmitted through the liquid 102 and the anterior chamber 13 exits through the exit window 104 in a direction perpendicular thereto. The size and shape of the container 101 are not limited as long as the light incident position on the periphery of the anterior chamber 13 of the eyeball 10 (for example, the cornea 14) is immersed in the liquid 102.

The immersion unit 100 serves to reduce the amount of change in the direction of the light reflected by the mirror 27 due to refraction of the light at the surface of the cornea 14. In other words, the influence of, for example, the shape of the cornea 14 may be reduced, and the optical path that passes through the anterior chamber 13 may be easily set. Light that travels along the optical path β is not reflected by the surface of the cornea 14.

The immersion unit 100 may be applied to the optical measurement apparatuses 1 according to other exemplary embodiments.

The eyeball observation unit 40 may observe the optical path relative to the eyeball 10 from a position in front of the eyeball 10 as described in the first exemplary embodiment. Alternatively, the beam splitter 70 may be used in place of the movable mirror 80 to observe the optical path relative to the eyeball 10 from a position shifted in the left-right direction from the position in front of the eyeball 10 as described in the second exemplary embodiment.

The optical measurement apparatus 1 according to the seventh exemplary embodiment may use the method of optical measurement of the eyeball 10 according to the first exemplary embodiment described with reference to FIG. 3.

### Eighth Exemplary Embodiment

In the eyeball optical measurement apparatuses 1 according to the fourth to seventh exemplary embodiments, the mirror 27 is set to a predetermined incident angle. In addition, the mirror 27 is spaced from the eyeball 10.

In an eighth exemplary embodiment, the mirror 27 is included in a contact member 110 that is brought into contact with the surface of the eyeball 10 when used.

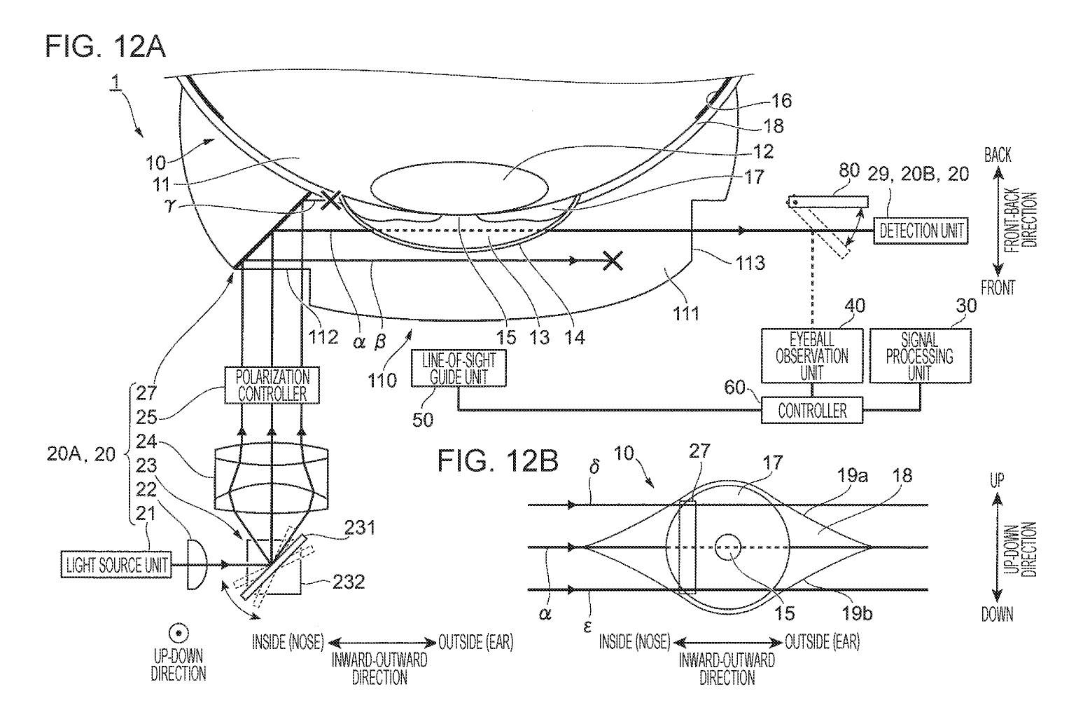

FIGS. 12A and 12B illustrate an exemplary structure of an eyeball optical measurement apparatus 1 according to the eighth exemplary embodiment. FIG. 12A shows a top view of the eyeball 10 (sectional view along a plane perpendicular to the up-down direction), and FIG. 12B shows a front view of the eyeball 10. The structure of the optical measurement apparatus 1 is similar to that in the fifth exemplary embodiment illustrated in FIGS. 8A and 8B except for the contact member 110 including the mirror 27 described below. Therefore, similar elements are denoted by the same reference numerals, and description thereof will be omitted. Differences from the fifth exemplary embodiment will now be described.

As illustrated in FIG. 12A, the contact member 110 is a component for an eyeball similar to a contact lens, and is placed on a surface of the cornea 14 of the eyeball 10 (eyeball surface). Here, the expression “the contact member 110 is placed on the eyeball 10” means that the contact member 110 is placed on the surface of the cornea 14 of the eyeball 10 (eyeball surface).

The contact member 110 includes a base body 111 and the mirror 27 disposed in the base body 111.

The base body 111 is made of, for example, a resin, such as poly(hydroxyethyl methacrylate), poly(methyl methacrylate), a silicone copolymer, or a fluorine-containing compound. When the refractive index of the base body 111 is close to the refractive indices of the aqueous humor in the anterior chamber 13 of the eyeball 10 and the cornea 14 of the eyeball 10, refraction at the interface between the contact member 110 and the eyeball 10 is reduced. Accordingly, the optical path that passes through the anterior chamber 13 of the eyeball 10 may be easily set. Light that travels along the optical path β is not reflected by the surface of the cornea 14.

A portion of the base body 111 through which the light travels toward the mirror 27 has a flat surface 112 that is perpendicular to the light. A portion of the base body 111 from which the light is emitted toward the detection unit 29 also has a flat surface 113 that is perpendicular to the light. Accordingly, when the light is incident on and emitted from the contact member 110 including the mirror 27, the optical path is not easily bent due to refraction caused by the base body 111.

As illustrated in FIG. 12B, the mirror 27 has a rectangular external shape. The mirror 27 may instead have other external shapes, such as an arc shape.

It is not necessary that the base body 111 be circular, and the base body 111 may instead have other shapes, such as a rectangular shape, as long as the base body 111 may be placed on the cornea 14.

The contact member 110 including the mirror 27 according to the eighth exemplary embodiment may be applied to the fourth to sixth exemplary embodiments.

The eyeball observation unit 40 may observe the optical path relative to the eyeball 10 from a position in front of the eyeball 10 as described in the first exemplary embodiment. Alternatively, the beam splitter 70 may be used in place of the movable mirror 80 to observe the optical path relative to the eyeball 10 from a position shifted in the left-right direction from the position in front of the eyeball 10 as described in the second exemplary embodiment.

The optical measurement apparatus 1 according to the eighth exemplary embodiment may use the method of optical measurement of the eyeball 10 according to the first exemplary embodiment described with reference to FIG. 3.

Although various exemplary embodiments have been described individually, the exemplary embodiments may be combined.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An eyeball optical measurement apparatus comprising:
a light radiating unit that radiates light toward an anterior chamber of an eyeball of a measurement subject;
a light receiving unit that receives the light that has passed through the anterior chamber;
an eyeball observation unit that observes an optical path relative to the eyeball while the light is radiated from the light radiating unit; and
a controller that controls the light radiating unit so as to change a position at which the eyeball is irradiated with the light on the basis of a result of the observation of the eyeball by the eyeball observation unit.

2. The eyeball optical measurement apparatus according to claim 1, wherein, when the optical path does not pass through the anterior chamber of the eyeball according to the result of the observation of the eyeball by the eyeball observation unit, the controller controls the light radiating unit so that the optical path passes through the anterior chamber of the eyeball.

3. The eyeball optical measurement apparatus according to claim 1, wherein the eyeball observation unit outputs image data representing a positional relationship between the eyeball and the optical path, and
wherein the controller controls the light radiating unit on the basis of the image data so that the eyeball and the optical path of the light are in a predetermined positional relationship.

4. The eyeball optical measurement apparatus according to claim 3, wherein the image data output by the eyeball observation unit is obtained by observing the eyeball from a position shifted leftward or rightward from a position in front of the eyeball.

5. The eyeball optical measurement apparatus according to claim 1, further comprising:
an optical-path switching unit that switches the light that has passed through the anterior chamber between an optical path toward the eyeball observation unit and an optical path toward the light receiving unit.

6. The eyeball optical measurement apparatus according to claim 1, wherein the controller controls the light radiating unit so that the optical path moves in one or both of a front-back direction of the eyeball and an up-down direction of the eyeball.

7. The eyeball optical measurement apparatus according to claim 6,
wherein the light radiating unit includes
a telecentric optical system that transmits the light, and
a mirror that is at a predetermined angle with respect to the telecentric optical system and that reflects the light toward the anterior chamber of the eyeball.

8. The eyeball optical measurement apparatus according to claim 1, further comprising:
a line-of-sight guide unit that guides a line of sight of the eyeball.

9. An eyeball optical measurement method comprising:
observing an optical path relative to an eyeball of a measurement subject while light is radiated toward the eyeball and acquiring first image data representing a positional relationship between the eyeball and the optical path;
performing an optical measurement on an anterior chamber of the eyeball;
observing the optical path relative to the eyeball and acquiring second image data representing the positional relationship between the eyeball and the optical path; and
processing a signal obtained by the optical measurement when a coincidence factor of the first image data and the second image data is less than or equal to a predetermined allowable value.
